(19) United States
(12) Patent Application Publication
(10) Pub. No.: US 2026/0045368 A1
(43) Pub. Date: Feb. 12, 2026

(54) CONSTRUCTION METHOD OF BENIGN-MALIGNANT PULMONARY NODULE DIFFERENTIAL DIAGNOSIS MODEL BASED ON SINGLE-CELL IMMUNE ATLAS

(71) Applicant: ZHEJIANG UNIVERSITY, Hangzhou (CN)

(72) Inventors: Yang XIA, Hangzhou (CN); Wen LI, Hangzhou (CN); Weiwei YIN, Hangzhou (CN); Hongyu SHI, Hangzhou (CN); Wei CHEN, Hangzhou (CN)

(73) Assignee: ZHEJIANG UNIVERSITY, Hangzhou (CN)

(21) Appl. No.: 19/356,329

(22) Filed: Oct. 13, 2025

Related U.S. Application Data
(63) Continuation of application No. PCT/CN2025/071658, filed on Jan. 10, 2025.

(30) Foreign Application Priority Data
Aug. 12, 2024 (CN) 202411096852.5

(51) Int. Cl. G16H 50/30; G01N 33/574; G01N 33/68; G16B 25/10; G16B 40/10; G16B 40/20

(52) U.S. Cl. CPC G16H 50/30; G01N 33/57492; G01N 33/6872; G16B 25/10; G16B 40/10; G16B 40/20

(57) ABSTRACT

A construction method for constructing a benign-malignant pulmonary nodule differential diagnosis model based on a single-cell immune atlas provided. In this method, PBMCs are obtained by utilizing peripheral blood samples, followed by CyTOF analysis to generate a CyTOF dataset. Using Phenotype Analysis and Representation Clustering (PARC) algorithm, cells are categorized into distinct phenotypes based on marker expression, and the frequencies of cell subsets are employed as potential modeling features that are finally selected by using the RF method with 10-fold cross-validation strategy, thereby enabling lung cancer screening and early diagnosis. This technology is characterized by its non-invasiveness, high sensitivity, and high specificity, improving the diagnostic accuracy of lung cancer screening and providing patients with earlier treatment opportunities and more suitable surgical approaches.

### CROSS-REFERENCE TO RELATED APPLICATIONS

This application is a continuation of International Application No. PCT/CN2025/071658, filed on Jan. 10, 2025, which claims priority to Chinese Patent Application 202411096852.5, filed on Aug. 12, 2024. The disclosures of the above-referenced applications are hereby incorporated by reference in their entity.

### TECHNICAL FIELD

The present disclosure belongs to the medical field and more specifically relates to a method for constructing a benign-malignant pulmonary nodule differential diagnosis model based on single-cell immune atlas using mass cytometry (CyTOF) technology.

### BACKGROUND

Lung cancer is one of the malignancies with the highest incidence and mortality rates worldwide. The majority of patients (48%) already have distant metastases at initial diagnosis, with a 5-year relative survival rate of only about 8%. In contrast, patients diagnosed with early-stage lung cancer have a 5-year survival rate of up to 62%, but the detection rate is only around 20%. Comparatively, for Stage I cancer, the 5-year survival rate increases significantly, ranging from 68% to 92%. Undoubtedly, increasing the detection rate of lung cancer at curable stages (Stages 0, I, and II) is the most effective way to reduce lung cancer mortality. Improving early diagnosis rates is crucial for lowering mortality and enhancing prognosis.

However, due to the lack of clinical symptoms and sensitive technologies, detecting lung cancer in these early stages is challenging. Randomized controlled trials, including the U.S. National Lung Screening Trial (NLST) and the Dutch-Belgian NELSON trial, have shown that screening with low-dose CT (LDCT) significantly reduces lung cancer mortality. However, uncertainties remain regarding LDCT's clinical effectiveness and cost-efficiency, particularly in diagnosing part-solid nodules, which is the most difficult and challenging aspect. Another issue with LDCT screening is the high rate of false-positive nodule detection: among nodules detected in high-risk populations, most are benign. A large-scale NLST study from the U.S. revealed that while 24.2% of individuals undergoing LDCT screening had lung nodules detected, up to 96.4% of these positive nodules were later diagnosed as benign (false positives). Other studies have shown that the proportion of benign lung nodules after surgical resection is as high as 20%, and 38% after biopsy. Multiple studies confirm that LDCT screening detects nodules at an average rate of about 20%, with over 90% being benign. Excessive false positives may lead to overdiagnosis, overtreatment, wasted medical resources, and increased anxiety among screened individuals.

Imaging-based models such as the Mayo model (originally developed in 1997 by the Mayo Clinic for patients with nodules 4-30 mm, incorporating factors like age, smoking history, nodule diameter, spiculation, and location), the Veterans Association (VA) model, and the BROCK model have been used to determine nodule characteristics. Their area under the curve (AUC) ranges from 0.65 to 0.83, indicating limited accuracy. Moreover, models like the Mayo clinical lung cancer prediction model were developed based on Western patients and may not be fully applicable to Chinese patients, posing practical limitations.

Therefore, the key to diagnosing and treating pulmonary nodules lies in effectively differentiating and triaging them, rapidly determining their benign or malignant nature, resecting malignant nodules as early as possible, while avoiding unnecessary overtreatment and reducing the proportion of benign nodules subjected to surgical resection. To address this significant unmet clinical need, highly sensitive and specific methods are urgently required to accurately identify malignant pulmonary nodules.

On the other hand, the World Health Organization (WHO) Classification of Thoracic Tumors (5th edition, 2021) categorizes atypical adenomatous hyperplasia (AAH) and adenocarcinoma in situ (AIS) as glandular precursor lesions, excluding them from the scope of lung adenocarcinoma, while classifying minimally invasive adenocarcinoma (MIA) and invasive adenocarcinoma (IA) as two types of lung cancer with different degrees of invasiveness. The multidisciplinary classification of lung adenocarcinoma by the International Association for the Study of Lung Cancer/American Thoracic Society/European Respiratory Society indicates that MIA rarely spreads to regional lymph nodes or metastasizes, with a nearly 100% disease-free survival rate post-surgery. Thus, sublobar resection without systematic lymph node dissection is an option for MIA, whereas the surgical approach for IA patients must be tailored clinically. Therefore, a method to effectively distinguish between MIA and IA preoperatively is needed to plan surgical strategies in advance.

The human immune system is closely related to the initiation and progression of tumors. During tumor development, the tumor immune microenvironment (TME) also undergoes continuous changes. Tumor-induced immune alterations are not limited to the tumor itself but are often accompanied by systemic immune dysregulation, highlighting the complex interplay between tumors and peripheral immunity. Studies have shown that pulmonary tumors contain various types of infiltrating immune cells, which play crucial roles in tumor initiation and progression. Macrophages and T-cell populations exhibit potential interactions within the TME, and lung tumors are also rich in other myeloid components, including neutrophils, non-classical monocytes, and intermediate monocytes. Meanwhile, research indicates that the presence of B cells is associated with protective immunity in lung cancer patients. Additionally, some clinical studies have demonstrated that a high density of tumor-infiltrating T cells correlates with increased median survival in cancer patients. Therefore, comprehensive and meticulous monitoring of peripheral immune status may provide significant assistance in distinguishing benign from malignant pulmonary nodules.

In recent years, CyTOF technology has combined traditional flow cytometry analysis with mass spectrometry detection, using metal isotope tags instead of fluorescent labels and quantifying the tags via mass spectrometry. This enables simultaneous detection of over 40 target proteins at the single-cell level without cross-channel interference or complex compensation calculations, significantly enhancing the ability to assess complex cellular systems and processes. It serves as a multiparameter, high-throughput single-cell protein detection platform. By detecting multi-marker combinations, CyTOF technology can distinguish various cell subsets, construct cellular atlases of healthy or diseased states, comprehensively analyze intracellular signaling networks, and provide high-dimensional insights into human basal immune status, offering comprehensive information on immune cell composition, phenotype, and function.

### SUMMARY

In view of this, an objective of the present disclosure is to provide a method for constructing a benign-malignant pulmonary nodule differential diagnosis model based on a single-cell immune atlas, specifically utilizing CyTOF technology. Peripheral blood mononuclear cells (PBMCs) are obtained from peripheral blood samples via Ficoll isolation (Ficoll density gradient centrifugation), followed by CyTOF measurement to generate a CyTOF dataset. Using Phenotype Analysis and Representation Clustering (PARC) algorithm, cells are categorized into distinct phenotypes based on marker expression, and the frequencies of cell subset are employed as modeling features to construct the benign-malignant pulmonary nodule differential diagnosis model.

To achieve the above objective, the present disclosure adopts the following technical solution:
1. subjecting a peripheral blood sample to Ficoll isolation to obtain PBMCs, suspending the PBMCs in 5 ml pre-cooled fluorescence-activated cell sorting (FACS) buffer (1×PBS+0.5% BSA), centrifuging at 4° C. under 400×g for 5 min, discarding supernatant, and resuspending cell sediment in the buffer, performing cell counting and quality assessment of the PBMCs before a CyTOF analysis to ensure a count greater than 3×10⁶ and a viability rate exceeding 85%.
2. selecting 40 metal-conjugated antibodies as markers for cell labeling; washing the PBMCs with a PBS buffer and staining with 0.5 mM cisplatin, undergoing the cells to Fc receptor block and binding with the antibodies for 30 min, removing unbound antibodies via centrifugation, and fixing the PBMCs in a 200 μL intercalator solution overnight; washing the cells in distilled water and resuspending, adding into 20% EQ calibration beads solution, and performing further analysis by a mass cytometer. The 40 metal-conjugated antibody markers in step include CD45, CD3, CD56, TCR γ/δ, CD196 (CCR6), CD14, IgD, CD123 (IL-3Rα), CD85j (ILT2), CD19, CD25 (IL-2Rα), CD274 (PD-L1), CD278 (ICOS), CD39, CD27, CD24, CD45RA, CD86, CD28, CD197 (CCR7), CD11c, CD33, CD152 (CTLA-4), CD161, CD185 (CXCR5), CD66b, CD183 (CXCR3), CD94, CD57, CD45RO, CD127 (IL-7Rα), CD279 (PD-1), CD38, CD194 (CCR4), CD20, CD16, HLA-DR, CD4, CD8a, CD11b.
3. classifying the cells into distinct phenotypes based on marker expression by Phenotype Analysis and Representation Clustering (PARC) algorithm, utilizing the frequencies of cell subsets as potential modeling features that are finally selected by using a RF method with 10-fold cross-validation strategy.
4. performing modeling through the RF to obtain the benign-malignant pulmonary nodule differential diagnosis model.

Optionally, the method further includes in step 3 that: selecting the cell frequencies of 34 immune cell subsets and markers as modeling features. The 19 features of the benign-malignant pulmonary nodule differential diagnosis model include: CD33⁻CD14⁻CD3⁺CD4⁺CD28⁺, CD33⁻CD14⁻CD3⁺CD4⁺CD274⁺, CD33⁻CD14⁻CD3⁺CD4⁺CD197⁺CD45RA⁺, CD33⁻CD14⁻CD3⁺CD4⁺HLA⁻DR⁺ CD38⁺, CD33⁻CD14⁻CD3⁺CD4⁺CXCR5⁻CD183⁻CCR6⁻, CD33⁻CD14⁻CD3⁺CD4⁺CD25⁺CD127⁻CD161⁻CD45RA⁺, CD33⁻CD14⁻CD3⁺CD8⁺CD197⁺CD45RA⁺, CD33⁻CD14⁻CD3⁻CD19⁺CD24⁺CD38⁺, CD33⁻CD14⁻CD3⁻CD20⁻CD38⁺CD27⁺, CD33⁻CD14⁻CD3⁻CD56⁺CD16⁺CD94, CD33⁻CD14⁻CD3⁻CD56⁺CD16⁺CD161⁺, CD3⁻CD19⁻CD56⁻CD14⁻CD123⁺CD11c⁺, CD33⁻CD14⁻CD3⁻CD56⁺CD16⁻, CD86, CD11c, CD183, CD94, CD4, CD11b; and 15 features of a lung cancer infiltration degree assessment model include: CD33⁻CD14⁻CD3⁺CD8⁺CD85j⁺, CD33⁻CD14⁻CD3⁺CD8⁺CD161⁺, CD33⁻CD14⁻CD3⁺CD4⁺, CD33⁻CD14⁺CD3⁺CD4⁺CD197⁻CD45RA⁺, CD33⁻CD14⁻CD3⁺CD4⁺HLA-DR⁺ CD38, CD33⁻CD14⁻CD3⁺CD4⁺HLA-DR⁺ CD38⁻, CD33⁻CD14⁻CD3⁺CXCR5⁺, CD33⁻CD14⁻CD3⁺CD8⁺CD197⁺CD45RA⁺; CD33⁻CD14⁻CD3⁻CD19⁺CD24⁺CD38⁺, CD33⁻CD14⁻CD3⁻CD56⁺CD16⁺CD57⁺, CD33⁻CD14⁻CD3⁻CD56⁺CD16⁺HLA-DR, CD3⁻CD19⁻CD56⁻CD14⁻HLA-DR⁺, CD56⁺.

Optionally, the method further includes in step 3 that: using a training set and a validation set, where the samples are classified into the training set and validation set according to chronological order of enrollment.

Optionally, the method further includes in step 2 that: employing a combination of 40 antibody markers.

Optionally, during sample enrollment in step 1, randomized grouping is adopted, covering various nodule sizes (e.g., ≤10 mm, 11-20 mm, 21-30 mm, etc.), various nodule types (e.g., solid nodule, part-solid nodule, pure ground-glass opacity nodule), and samples with various degrees of adenocarcinoma invasiveness (e.g., AAH, AIS, MIA, IA, etc.).

As revealed by the above technical solution, this disclosure discloses a method for constructing a single-cell immune atlas-based benign-malignant pulmonary nodule differential diagnosis model. It demonstrates excellent diagnostic performance in differential diagnosis of carcinoma (CA) (pathologically confirmed CA) from non-carcinoma (non-CA) samples (including imaging-confirmed non-CA and pathologically confirmed non-CA), with the AUCs of the training set and validation set reaching 0.95 and 0.96, respectively, outperforming existing clinically used models: the Mayo model (training set and validation set AUCs of 0.75 and 0.70), Veterans Association (VA) model (with the AUCs of the training set and validation set being 0.73 and 0.65 respectively), and BROCK model (with the AUCs of the training set and validation set being 0.84 and 0.85 respectively). Moreover, it excels even in the most challenging differentiation between pathological non-CA groups and CA groups, achieving the AUCs of the training set and validation set of 0.92 and 0.90, significantly surpassing current clinical models: the Mayo model (training set and validation set AUCs of 0.69 and 0.61), Veterans Association (VA) model (with the AUCs of the training set and validation set being 0.68 and 0.61 respectively), and BROCK model (with the AUCs of the training set and validation set being 0.72 and 0.65 respectively).

Additionally, the model constructed in this solution can effectively differentiate MIA from IA preoperatively, with the AUCs of the training set and the validation set being 0.97 and 0.93, respectively. This disclosure enhances the sensitivity and specificity of lung cancer screening, reduces the risk of missed and misdiagnosis, and can be applied to auxiliary diagnosis and screening for lung cancer.

The present disclosure provides a lung cancer screening technology based on CyTOF data for cellular immune profiling analysis. By detecting the expression profiles of tumor-associated immune cells in a patient's peripheral blood, classifying peripheral blood sample data, and combining artificial intelligence algorithms, a learning model is constructed. The peripheral blood sample data is input into this model to obtain a risk score indicating the likelihood of the patient having lung cancer, thereby achieving lung cancer screening and early diagnosis. This technology is non-invasive and characterized by high sensitivity and specificity, enabling rapid and accurate detection of lung cancer-related immune cell expression profiles in peripheral blood, which improves the diagnostic accuracy of lung cancer screening and provides patients with earlier treatment opportunities and optimal therapeutic approaches.

The advantages of the present disclosure include: (1) improving the sensitivity and specificity of lung cancer screening, reducing the risk of missed and misdiagnosis; (2) avoiding invasive diagnostic procedures, thereby lowering clinical risks; (3) enabling early diagnosis of lung cancer, offering patients earlier treatment opportunities; (4) further classifying the extent of lung cancer infiltration to provide more suitable surgical options for patients; (5) utilizing CyTOF technology, which features high throughput, high resolution, and high sensitivity, allowing rapid and accurate detection of lung cancer-related immune cell expression profiles; (6) incorporating artificial intelligence algorithms to enhance the accuracy and reliability of lung cancer screening.

### BRIEF DESCRIPTION OF DRAWINGS

To more clearly illustrate the technical solutions in the embodiments of the present disclosure or the prior art, the following briefly introduces the accompanying drawings required for describing the embodiments or the prior art. Obviously, the drawings in the following description are merely examples of the present disclosure. For those skilled in the art, other drawings can be obtained from the provided ones without creative effort.

FIG. 1 is a flowchart of the lung cancer screening based on CyTOF cellular immune profiling in the present disclosure.

FIG. 2 is a box plot of risk scores for CA and non-CA group samples in the training set and validation set of the lung cancer diagnostic model in the present disclosure.

FIG. 3 is an AUC plot of the training set and validation set of the lung cancer diagnostic model in the present disclosure.

FIG. 4 is a box plot of risk scores for the most challenging pathological non-CA group and CA group samples in the training set and validation set of the lung cancer diagnostic model in the present disclosure.

FIG. 5 is an AUC plot of the most challenging pathological non-CA group and CA group samples in the training set and validation set of the lung cancer diagnostic model in the present disclosure.

FIG. 6 is a box plot of risk scores for MIA and non-IA group samples in the training set and validation set of the surgical decision model in the present disclosure.

FIG. 7 is an AUC plot of the training set and validation set of the surgical decision model in the present disclosure.

### DESCRIPTION OF EMBODIMENTS

The technical solutions of the present disclosure are described clearly and completely below in conjunction with the accompanying drawings and embodiments. It is evident that the described embodiments are only a part of the embodiments of the present disclosure, rather than all of them. Based on the embodiments of the present disclosure, all other embodiments obtained by those of ordinary skill in the art without creative effort shall fall within the scope of protection of the present disclosure.

### Example 1: a Method for Constructing a Benign-Malignant Pulmonary Nodule Differential Diagnosis Model Based on Single-Cell Immune Atlas Using CyTOF

The specific steps are as follows:

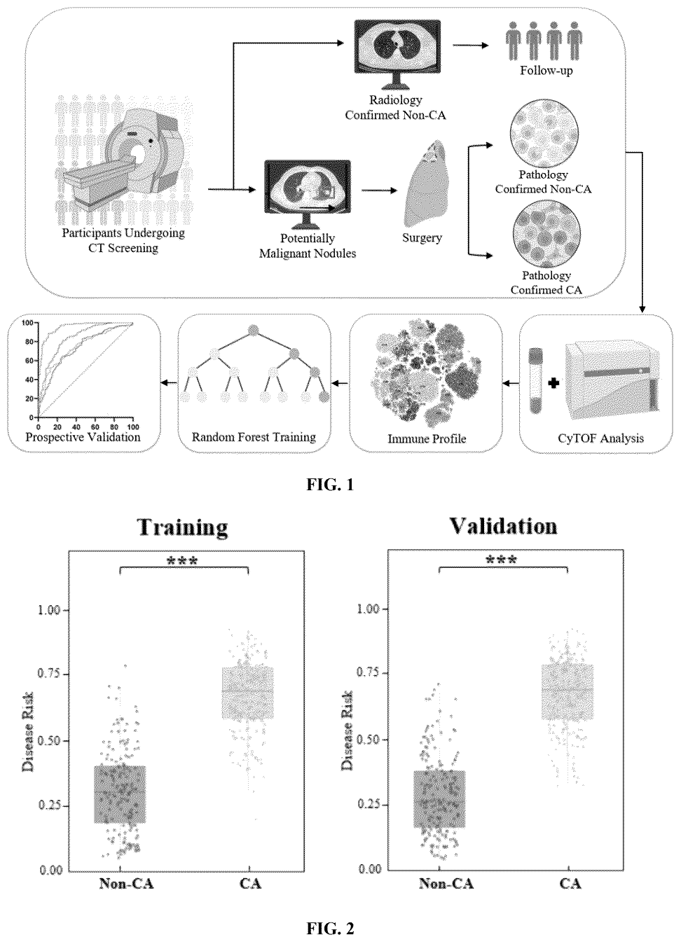

1. Peripheral blood samples (5 ml per case) were collected and delivered to the laboratory for further processing within 12 hours at room temperature or 48 hours at 4° C. low-temperature conditions. Subjects were required to meet the following inclusion criteria: age ≥18 years; (patients with pulmonary nodules scheduled for surgical resection confirmed by histopathology, those with pulmonary nodules showing no changes after 3-year follow-up, or pulmonary nodules ≤4 mm in diameter); and signed informed consent. Exclusion criteria were defined as: history of cancer treatment; acute infection phase; blood transfusion within 6 months prior to sampling; use of medications affecting peripheral blood components within 2 weeks prior to sampling; locally recurrent tumors; organ decompensation; immunodeficiency syndrome; hematologic precancerous conditions; immunosuppressive therapy; or coagulation disorders. A total of 1,032 peripheral blood samples were obtained for this project. As shown in FIG. 1, the collected peripheral blood samples were processed as follows upon transfer to the laboratory.

2. Sample preprocessing, including: PBMCs were isolated from the blood by Ficoll-Paque density gradient centrifugation. The cells were suspended in a 5 ml pre-cooled FACS buffer (1×PBS+0.5% BSA), centrifuged at 4° C. under 400×g for 5 mins, discarded supernatant, and resuspended cell sediment in the buffer. Cell counting and quality assessment of the PBMCs were performed before a CyTOF analysis to ensure a count greater than 3×10⁶ and a viability rate above 85%.

3. CyTOF staining and data analysis, including: 40 metal-conjugated antibodies were selected as markers for cell labeling; the PBMCs were washed with a PBS buffer and stained with 0.5 mM cisplatin, the cells were blocked with Fc receptors and bound with the antibodies for 30 min. Unbound antibodies were removed via centrifugation. The PBMCs were fixed in a 200 μL intercalation solution. The cells were washed in distilled water and resuspended, added into 20% EQ calibration beads solution, and performed further analyzed by a mass cytometer. FCS files were normalized using the bead normalization method. Each sample dataset was debarcoded using a dual-state filtering scheme with unique mass-tag barcodes. FlowJo software was employed to exclude debris, dead cells, and doublets, retaining only live single immune cells.

4. Feature selection: the samples were classified into a training set and validation set according to chronological order of enrollment. The training set included 178 lung cancer samples and 218 non-CA controls. First, the negative and positive expression of markers on each cell in the training set were evaluated. Then, based on the expression profiles of these markers, the Random Forest (RF) algorithm and 10-fold cross-validation were used to select characteristic cell subsets. Characteristics with an importance level exceeding 0.01 in each successful random forest model constructions were recorded. If a characteristic appeared over 350 times in 1000 cross-validation iterations, it was counted. Ultimately, 19 characteristic cell subsets were screened for model construction.

5. Model construction: using the 178 lung cancer samples and 218 non-CA controls from the training set, the characteristics selected above were employed to build a lung cancer diagnostic model via the RF. Risk scores for each participants were calculated by the modeling, which representing the average probability of a sample being judged positive by each decision tree in the random forest model, ranging from 0 to 1.

### Example 2: a Validation Method for a Benign-Malignant Pulmonary Nodule Differential Diagnosis Model Based on Single-Cell Immune Atlas Using CyTOF Technology

1. Following the protocol provided in Example 1, CyTOF staining and data analysis were performed on the validation set, which included 251 untrained non-CA samples and 283 lung cancer samples.

2. New peripheral blood samples were input into the lung cancer diagnostic model constructed with the 19 cell subsets for prediction and evaluation of the blood samples. It was determined whether the sample was from a lung cancer patient based on the prediction results of the lung cancer diagnostic model.

### Example 3: Construction and Validation of a Model for Assessing the Invasive Degree of Lung Cancer Based on Single-Cell Immune Atlas Using CyTOF

1. Following the method provided in Example 1, CyTOF staining and data analysis were performed by using 113 pulmonary nodule samples pathologically diagnosed as MIA and 105 pulmonary nodule samples pathologically diagnosed as IA in the training set.

2. Following the method provided in Example 1, the RF and 10-fold cross-validation were employed to select characteristic cell subsets. A total of 15 cell subsets were screened as modeling features to construct a new model for determining the invasive degree of malignant pulmonary nodules.

3. Following the method provided in Example 2, model validation was conducted by using 111 pulmonary nodule samples pathologically diagnosed as MIA and 106 pulmonary nodule samples pathologically diagnosed as IA in the validation set.

Clearly, the above Examples of the present disclosure are merely illustrative examples provided to explain the present disclosure more clearly and do not limit the implementation of the present disclosure. For those skilled in the art, other variations or modifications in different forms may be made based on the above description. It is impossible to exhaust all implementation methods here, and any obvious variations or modifications derived from the technical solutions of the present disclosure still fall within the protection scope of the present disclosure.

Referring to FIGS. 2-3, it can be seen that the benign-malignant pulmonary nodule differential diagnosis model disclosed in the present disclosure exhibits excellent diagnostic performance in distinguishing lung cancer (pathologically confirmed CA) from non-CA samples (including imaging-confirmed non-CA and pathologically confirmed non-CA). The AUCs of the training set and the validation set reached 0.95 and 0.96, respectively, outperforming existing clinically used models: the Mayo model (with the AUCs of the training set and validation set being 0.75 and 0.70 respectively), the Veterans Association (VA) model (with the AUCs of the training set and validation set being 0.73 and 0.65 respectively), and the BROCK model (with the AUCs of the training set and validation set being 0.84 and 0.85 respectively).

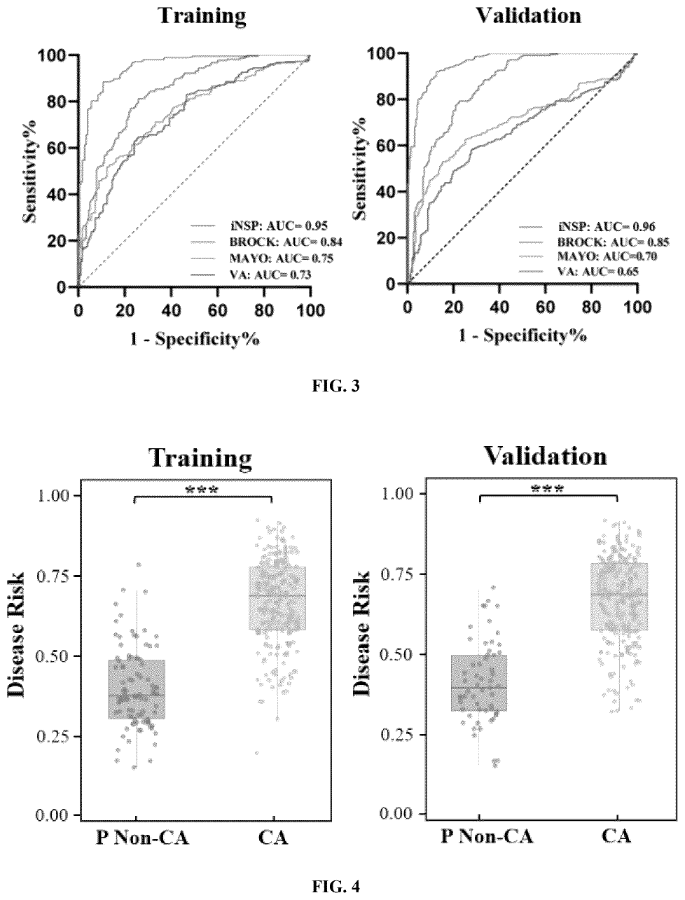

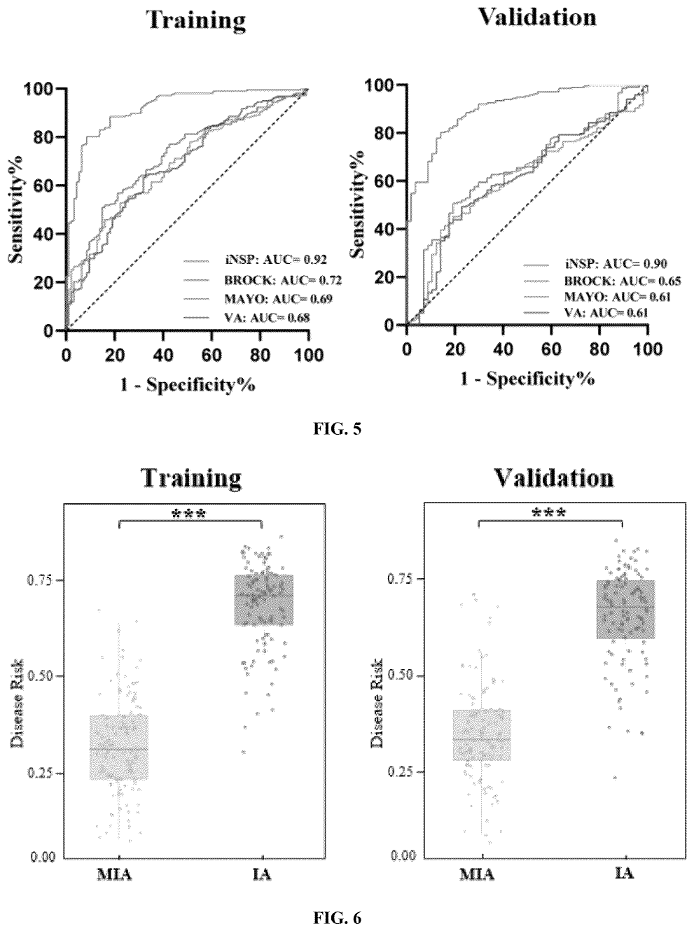

Referring to FIGS. 4-5, it can be seen that the benign-malignant pulmonary nodule differential diagnosis model disclosed in the present disclosure demonstrates outstanding performance in the most challenging differentiation between pathologically non-CA and lung cancer groups (with the AUCs of the training set and validation set being 0.92 and 0.90 respectively), surpassing existing clinically used models: the Mayo model, the Veterans Association (VA) model, and the BROCK model.

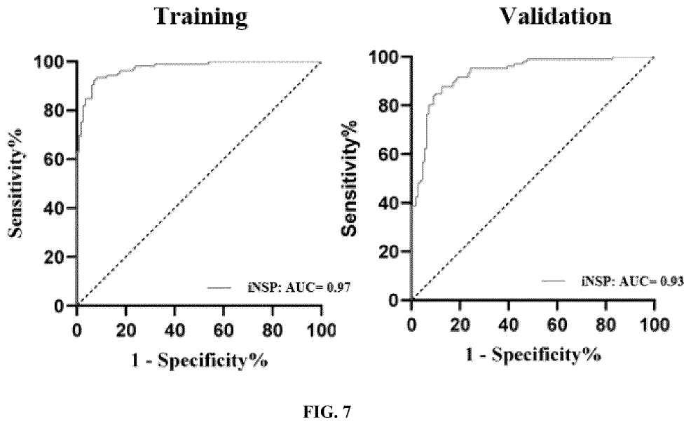

Referring to FIGS. 6-7, the model constructed in this solution can effectively distinguish MIA from IA preoperatively, with the AUCs of the training set and validation set being 0.97 and 0.93, respectively.

The various Examples in this specification are described in a progressive manner, with each Example focusing on the differences from other embodiments. Similar or identical parts between the Examples can be cross-referenced. As for the steps disclosed in the embodiments, since they correspond to the methods disclosed in the Examples, the description is relatively brief, and relevant details can be referred to in the method section.

The above description of the disclosed embodiments enables those skilled in the art to implement or use the present disclosure. Various modifications to these Examples will be apparent to those skilled in the art, and the general principles defined herein may be applied to other Examples without departing from the spirit or scope of the present disclosure. Therefore, the present disclosure is not limited to the Examples shown herein but is to be accorded the widest scope consistent with the principles and novel features disclosed herein.

What is claimed is:

1. A construction method for constructing a benign-malignant pulmonary nodule differential diagnosis model based on a single-cell immune atlas, comprising the following steps: obtaining PBMCs from peripheral blood samples by using a Ficoll isolation method, performing a CyTOF measurement on the PBMCs to obtain a CyTOF dataset, employing Phenotype Analysis and Representation Clustering algorithm to classify the cells into distinct phenotypes based on marker expression, and utilizing the frequencies of cell subsets as modeling features to obtain the benign-malignant pulmonary nodule differential diagnosis model.

2. The construction method according to claim 1, wherein the method is realized through the following:
Step a. subjecting a peripheral blood sample to Ficoll isolation to obtain PBMCs, suspending the PBMCs in 5 ml pre-cooled fluorescence-activated cell sorting buffer, centrifuging at 4° C. under 400×g for 5 min, discarding supernatant, and resuspending cell sediment in the buffer, performing cell counting and quality assessment of the PBMCs before a CyTOF running to ensure a count greater than 3×10⁶ and a viability rate exceeding 85%;
Step b. selecting 40 metal-conjugated antibodies as markers for cell labeling; washing the PBMCs with a PBS buffer and staining with 0.5 mM cisplatin, undergoing the cells to Fc receptor block and binding with the antibodies for 30 min, removing unbound antibodies via centrifugation, and fixing the PBMCs in a 200 μL intercalation solution overnight; washing the cells in distilled water and resuspending, adding into 20% EQ calibration beads solution, and performing further analysis by a mass cytometer;
Step c. classifying the cells into distinct phenotypes based on marker expression by using Phenotype Analysis and Representation Clustering algorithm, utilizing the frequencies of cell subsets as potential modeling features that are finally selected by using the RF method with 10-fold cross-validation strategy; and
Step d. performing modeling through the RF to obtain the benign-malignant pulmonary nodule differential diagnosis model.

3. The construction method according to claim 2, wherein in step a, for enrollment of the peripheral blood sample, randomized grouping is adopted, covering various nodule sizes, various nodule types, and samples with various degrees of adenocarcinoma invasiveness.

4. The construction method according to claim 3, wherein the various nodule sizes comprise ≤10 mm, 11-20 mm and 21-30 mm, the various nodule types comprise solid nodule, part-solid nodule, and pure ground-glass opacity nodule, and the samples with various degrees of adenocarcinoma invasiveness comprise AAH, AIS, MIA, and IA.

5. The construction method according to claim 2, wherein the fluorescence-activated cell sorting buffer in step a is 1×PBS+0.5% BSA.

6. The construction method according to claim 2, wherein the markers of the 40 metal-conjugated antibody in step b comprise: CD45, CD3, CD56, TCR γ/δ, CD196, CD14, IgD, CD123, CD85j, CD19, CD25, CD274, CD278, CD39, CD27, CD24, CD45RA, CD86, CD28, CD197, CD11c, CD33, CD152, CD161, CD185, CD66b, CD183, CD94, CD57, CD45RO, CD127, CD279 (PD-1), CD38, CD194, CD20, CD16, HLA-DR, CD4, CD8a, CD11b.

7. The construction method according to claim 2, wherein a combination of the markers of the 40 metal-conjugated antibody is employed in step b.

8. The construction method according to claim 2, wherein frequencies of 34 immune cell subsets and markers are selected in step c as modeling features, which comprise 19 features of benign-malignant pulmonary nodule differential diagnosis model comprising: CD33⁻CD14⁻CD3⁺CD4⁺CD28⁺, CD33⁻CD14⁻CD3⁺CD4⁺CD274⁺, CD33⁻CD14⁻CD3⁺CD4⁺CD197⁺CD45RA⁺, CD33⁻CD14⁻CD3⁺CD4⁺HLA-DR⁺CD38⁺, CD33⁻CD14⁻CD3⁺CD4⁺CXCR5⁻CD183⁻CCR6, CD33⁻CD14⁻CD3⁺CD4⁺CD25⁺CD127⁻CD161⁻CD45RA⁺, CD33⁻CD14⁻CD3⁺CD8⁺CD197⁺CD45RA⁺, CD33⁻CD14⁻CD3⁻CD19⁺CD24⁺CD38⁺, CD33⁻CD14⁻CD3⁻CD20⁻CD38⁺CD27⁺, CD33⁻CD14⁻CD3⁻CD56⁺CD16⁺CD94⁺, CD33⁻CD14⁻CD3⁻CD56⁺CD16⁺CD161⁺, CD3⁻CD19⁻CD56⁻CD14⁻CD123⁺CD11c⁺, CD33⁻CD14⁻CD3⁻CD56⁺CD16, CD86, CD11c, CD183, CD94, CD4, CD11b; and 15 features of lung cancer invasiveness assessment model comprising: CD33⁻CD14⁻CD3⁺CD8⁺CD85j⁺, CD33⁻CD14⁻CD3⁺CD8⁺CD161⁺, CD33⁻CD14⁻CD3⁺CD4⁺, CD33⁻CD14⁻CD3⁺CD4⁺CD197⁻CD45RA⁺, CD33⁻CD14⁻CD3⁺CD4⁺HLA-DR⁺CD38⁺, CD33⁻CD14⁻CD3⁺CD4⁺HLA-DR⁺CD38⁺, CD33⁻CD14⁻CD3⁺CXCR5⁺, CD33⁻CD14⁻CD3⁺CD8⁺CD197⁺CD45RA; CD33⁻CD14⁻CD3⁻CD19⁺CD24⁺CD38⁺, CD33⁻CD14⁻CD3⁻CD56⁺CD16⁺CD57⁺, CD33⁻CD14⁻CD3⁻CD56⁺CD16⁺HLA-DR⁺, CD3⁻CD19⁻CD56⁻CD14⁻HLA-DR⁻, CD56.

9. The construction method according to claim 2, wherein a training set and a validation set are used in step c, wherein the samples are classified into the training set and validation set according to chronological order of enrollment.
